(19) United States
(12) Patent Application Publication
(10) Pub. No.: US 2018/0169602 A1
(43) Pub. Date: Jun. 21, 2018

(54) UPGRADING HYDROCARBONS USING STOICHIOMETRIC OR BELOW STOICHIOMETRIC AIR FOR CATALYST REGENERATION

(71) Applicant: ExxonMobil Research and Engineering Company, Annandale, NJ (US)

(72) Inventors: Mohsen N. Harandi, New Hope, PA (US); Alvin U. Chen, The Woodlands, TX (US); Charles R. Bolz, West Chester, PA (US); Christopher G. Smalley, Lanoka Harbor, NJ (US); Masaaki Sugita, The Woodlands, TX (US); Suriyanarayanan Rajagopalan, Spring, TX (US); Bing Du, Pittstown, NJ (US)

(21) Appl. No.: 15/825,174

(22) Filed: Nov. 29, 2017

Related U.S. Application Data
(60) Provisional application No. 62/434,447, filed on Dec. 15, 2016.

(51) Int. Cl. B01J 8/26; B01J 8/18; B01J 29/40; C07C 2/12

(52) U.S. Cl. CPC B01J 8/26; B01J 8/1872; C10G 2300/1092; C07C 2/12; C10G 2400/02; B01J 29/40

(57) ABSTRACT

A method is provided for upgrading a hydrocarbon feed. The method may include contacting a hydrocarbon feed with a catalyst in a fluidized bed reactor; directing a portion of the catalyst from the fluidized bed reactor to a regeneration zone, such that the portion of the catalyst flows in a first direction through the regeneration zone; directing combustion air into the regeneration zone in a counter-flow direction to the first direction, wherein the combustion air is provided at a rate of about 100.05% or less of the stoichiometric air requirement for combusting coke present on the portion of catalyst; regenerating the portion of the catalyst in the regeneration zone to produce regenerated catalyst; and directing a portion of the hydrocarbon feed to combine with the regenerated catalyst downstream of the regeneration zone and lift the regenerated catalyst back to the fluidized bed reactor.

### CROSS-REFERENCE TO RELATED APPLICATIONS

This application claims priority to U.S. Provisional Application Ser. No. 62/434,447 filed Dec. 15, 2016, which is herein incorporated by reference in its entirety.

This application is related to a co-pending U.S. application, filed on even date herewith, identified under Attorney Docket number 2016EM362-US2, entitled “Upgrading Fuel Gas Using Stoichiometric Air For Catalyst Regeneration,” hereby incorporated by reference herein in its entirety.

### FIELD

This application relates to the field of hydrocarbon upgrading using a fluidized bed reactor with an integrated catalyst regenerator.

### BACKGROUND

Hydrocarbon feeds such as fuel gas and naphtha-containing feeds are sometimes upgraded in fluidized bed reactors that incorporate regenerators for regenerating catalysts used in upgrading the hydrocarbons.

For example, olefin-containing fuel gas may be upgraded to gasoline using a Mobil Olefins to Gasoline (“MOG”) process. In this process, a fluidized bed reactor containing a catalyst receives the fuel gas feed and oligomerizes olefins in the fuel gas to produce C5+ gasoline. Catalyst particles are circulated to a regenerator to burn the coke that is formed during the oligomerization reactions. Typically, multiple times the stoichiometric air requirement (i.e., a theoretical excess amount of air) is fed to the regenerator to maintain the desired superficial velocity in the regenerator, to achieve a desirable vessel diameter, and to achieve the complete combustion of coke. The reason this is so, is because the coke make in the process, as a % of the weight of the feed olefins is less compared to conventional fluid catalytic cracking.

In another example, naphtha-containing feeds may be upgraded to reduce the sulfur content of the feed in processes that utilize a fluidized bed reactor with a similar regeneration scheme. These regenerators also typically reqiure multiple times the stoichiometric air requirement to be fed to the regenerator for the same reasons, e.g., to maintain the desired superficial velocity in the regenerator, to achieve a desirable vessel diameter, and to achieve the complete combustion of coke.

It would therefore be desirable to provide new processes and systems for upgrading hydrocarbons that may be operated with stoichiometric or below stoichiometric air feeds or that may otherwise overcome one or more of the drawbacks of the current pocesses.

### SUMMARY

In one aspect, a method is provided for upgrading a hydrocarbon feed. The method may include contacting a hydrocarbon feed with a catalyst in a fluidized bed reactor; directing a portion of the catalyst from the fluidized bed reactor to a regeneration zone, such that the portion of the catalyst flows in a first direction through the regeneration zone; directing combustion air into the regeneration zone in a counter-flow direction to the first direction, wherein the combustion air is provided at a rate of about 100.05% or less of the stoichiometric air requirement for combusting coke present on the portion of catalyst; regenerating the portion of the catalyst in the regeneration zone to produce regenerated catalyst; and directing a portion of the hydrocarbon feed to combine with the regenerated catalyst downstream of the regeneration zone and lift the regenerated catalyst back to the fluidized bed reactor.

In another aspect, a system is provided for upgrading a fuel gas. The system includes a fuel gas feed; a fluidized bed reactor containing a catalyst for converting the fuel gas to gasoline boiling range hydrocarbons; a regeneration leg fluidly connected with the fluidized bed reactor for receiving a portion of catalyst to be regenerated and permit the portion of catalyst to flow in a first direction through a regeneration zone; a combustion air feed fluidly connected with the regeneration leg and adapted to inject combustion air into the regeneration leg so that the combustion air flows through the regeneration zone in a counter-flow direction to the first direction to produce a regenerated catalyst; and a lift leg fluidly connected to the regeneration leg to receive the regenerated catalyst, the lift leg further fluidly connected to the fuel gas feed to receive a portion of the fuel gas feed to lift the regeneration catalyst away from the regeneration leg and return the regenerated catalyst to the fluidized bed reactor.

### DRAWINGS

FIG. 1 is a schematic illustrating an exemplary process of regeneration of catalyst according to one or more embodiments of the present invention.

### DETAILED DESCRIPTION

Systems and methods are provided for catalyst regeneration using a stoichiometric amount or less air for coke combustion. Such a system and method may allow for reduction in air compressor, start-up heater demands and sizes, reduction in regenerator size and the strutural demand to accommodate the regenerator.

These and other advantages may be achieved by contacting a hydrocarbon feed with a catalyst in a fluidized bed reactor; directing a portion of the catalyst from the fluidized bed reactor to a regeneration zone, such that the portion of the catalyst flows in a first direction through the regeneration zone; directing combustion air into the regeneration zone in a counter-flow direction to the first direction, wherein the combustion air is provided at a rate of about 120% or less, preferably 100.05% or less (preferably less than 100%) of the stoichiometric air requirement for combusting coke present on the portion of catalyst; regenerating the portion of the catalyst in the regeneration zone to produce regenerated catalyst; and directing a portion of the hydrocarbon feed to combine with the regenerated catalyst downstream of the regeneration zone and lift the regenerated catalyst back to the fluidized bed reactor.

The hydrocarbon feed may be any type of hydrocarbon feed, such as a fuel gas to be upgraded to gasoline boiling range hydrocarbons or a fluid catalytic cracking naphtha to be desulfurized. The catalyst can be any catalyst to be regenerated used in such processes, such as ZSM-5.

The regenerated catalyst upon regeneration may be fed to a lift leg where it combines with the portion of the fuel gas. The regenerated catalyst may be fed through the regeneration zone and to the lift leg by force of gravity. The regeneration zone may be a vertically-oriented conduit and the first direction may be a vertical direction.

The portion of the hydrocarbon feed that is fed to the lift leg to transport the regenerated catalyst to the fluidized bed reactor may be less than about 20 wt % of the hydrocarbon feed fed to the fluidized bed reactor, such as between about 5 wt % and about 10 wt % of the hydrocarbon feed fed to the fluidized bed reactor.

Byproducts of the combustion of coke may be directed into the fluidized bed reactor, for example, into a stripping section within the fluidized bed reactor.

As used herein, and unless specified otherwise, “gasoline” or “gasoline boiling range hydrocarbons” refers to a composition containing at least predominantly C5-C12 hydrocarbons. In one embodiment, gasoline or gasoline boiling range components is further defined to refer to a composition containing at least predominantly C5-C12 hydrocarbons and further having a boiling range of from about 100° F. to about 400° F. In an alternative embodiment, gasoline or gasoline boiling range components is defined to refer to a composition containing at least predominantly C5-C12 hydrocarbons, having a boiling range of from about 100° F. to about 400° F., and further defined to meet ASTM standard D439.

### Hydrocarbon Feeds

The present processes and systems may be employed with various hydrocarbon feeds; however, the processes and systems disclosed herein are particularly useful in upgrading fuel gas to gasoline range hydrocarbons. For example, the hydrocarbon feed may be a fuel gas comprising C5− hydrocarbons, particularly fuel gas feedstreams comprising C4 and lighter hydrocarbons, including feedstreams that are predominantly C3 hydrocarbons or feedstreams that comprise C2-hydrocarbons.

The present processes and systems may also be employed with the regeneration of catalysts for desulfurization, such as those used in fluidized reactor beds to remove sulfur from naphtha streams produced by fluid catalytic cracking units.

### Reaction System

In various aspects, the hydrocarbon feed can be exposed to an acidic catalyst (such as a zeolite) under effective conversion conditions for olefinic oligomerization and/or sulfur removal. Optionally, the zeolite or other acidic catalyst can also include a hydrogenation functionality, such as a Group VIII metal or other suitable metal that can activate hydrogenation/dehydrogenation reactions. The hydrocarbon feed can be exposed to the acidic catalyst without providing substantial additional hydrogen to the reaction environment. Added hydrogen refers to hydrogen introduced as an input flow to the process, as opposed to any hydrogen that might be generated in-situ during processing. Exposing the feed to an acidic catalyst without providing substantial added hydrogen is defined herein as exposing the feed to the catalyst in the presence of a) less than about 100 SCF/bbl of added hydrogen, or less than about 50 SCF/bbl; b) a partial pressure of less than about 50 psig (350 kPag), or less than about 15 psig (100 kPag) of hydrogen; or c) a combination thereof.

The acidic catalyst used in the processes described herein can be a zeolite-based catalyst, that is, it can comprise an acidic zeolite in combination with a binder or matrix material such as alumina, silica, or silica-alumina, and optionally further in combination with a hydrogenation metal. More generally, the acidic catalyst can correspond to a molecular sieve (such as a zeolite) in combination with a binder, and optionally a hydrogenation metal. Molecular sieves for use in the catalysts can be medium pore size zeolites, such as those having the framework structure of ZSM-5, ZSM-11, ZSM-12, ZSM-22, ZSM-23, ZSM-35, ZSM-48, or MCM-22. Such molecular sieves can have a 10-member ring as the largest ring size in the framework structure. The medium pore size zeolites are a well-recognized class of zeolites and can be characterized as having a Constraint Index of 1 to 12. Constraint Index is determined as described in U.S. Pat. No. 4,016,218 incorporated herein by reference. Catalysts of this type are described in U.S. Pat. Nos. 4,827,069 and 4,992,067 which are incorporated herein by reference and to which reference is made for further details of such catalysts, zeolites and binder or matrix materials.

Additionally or alternately, catalysts based on large pore size framework structures (12-member rings) such as the synthetic faujasites, especially zeolite Y, such as in the form of zeolite USY. Zeolite beta may also be used as the zeolite component. Other materials of acidic functionality which may be used in the catalyst include the materials identified as MCM-36 and MCM-49. Still other materials can include other types of molecular sieves having suitable framework structures, such as silicoaluminophosphates (SAPOs), aluminosilicates having other heteroatoms in the framework structure, such as Ga, Sn, or Zn, or silicoaluminophosphates having other heteroatoms in the framework structure. Mordenite or other solid acid catalysts can also be used as the catalyst.

In various aspects, the exposure of the hydrocarbon feed to the acidic catalyst can be performed in any convenient manner, such as exposing the hydrocarbon feed to the acidic catalyst under fluidized bed conditions, moving bed conditions, and/or in a riser reactor. In some aspects, the particle size of the catalyst can be selected in accordance with the fluidization regime which is used in the process. Particle size distribution can be important for maintaining turbulent fluid bed conditions as described in U.S. Pat. No. 4,827,069 and incorporated herein by reference. Suitable particle sizes and distributions for operation of dense fluid bed and transport bed reaction zones are described in U.S. Pat. Nos. 4,827,069 and 4,992,607 both incorporated herein by reference. Particle sizes in both cases will normally be in the range of 10 to 300 microns, typically from 20 to 100 microns.

Acidic zeolite catalysts suitable for use as described herein can be those exhibiting high hydrogen transfer activity and having a zeolite structure of ZSM-5, ZSM-11, ZSM-12, ZSM-22, ZSM-23, ZSM-35, ZSM-48, MCM-22, MCM-36, MCM-49, zeolite Y, and zeolite beta. Such catalysts can be capable of oligomerizing olefins from the hydrocarbon feed. For example, such catalysts can convert C2-C4 olefins, such as those present in a refinery fuel gas, to C5+ olefins. Such catalysts can also be capable of converting organic sulfur compounds such as mercaptans to hydrogen sulfide without added hydrogen by utilizing hydrogen present in the hydrocarbon feed. Group VIII metals such as nickel may be used as desulfurization promoters. A fluid-bed reactor/regenerator can assist with maintaining catalyst activity in comparison with a fixed-bed system. Further, the hydrogen sulfide produced in accordance with the processes described herein can be removed using conventional amine based absorption processes.

ZSM-5 crystalline structure is readily recognized by its X-ray diffraction pattern, which is described in U.S. Pat. No. 3,702,866. ZSM-11 is disclosed in U.S. Pat. No. 3,709,979, ZSM-12 is disclosed in U.S. Pat. No. 3,832,449, ZSM-22 is disclosed in U.S. Pat. No. 4,810,357, ZSM-23 is disclosed in U.S. Pat. Nos. 4,076,842 and 4,104,151, ZSM-35 is disclosed in U.S. Pat. No. 4,016,245, ZSM-48 is disclosed in U.S. Pat. No. 4,375,573 and MCM-22 is disclosed in U.S. Pat. No. 4,954,325. The U.S. Patents identified in this paragraph are incorporated herein by reference.

While suitable zeolites having a coordinated metal oxide to silica molar ratio of 20:1 to 200:1 or higher may be used, it can be advantageous to employ aluminosilicate ZSM-5 having a silica:alumina molar ratio of about 25:1 to 70:1, suitably modified. A typical zeolite catalyst component having Bronsted acid sites can comprises, consist essentially of, or consist of crystalline aluminosilicate having the structure of ZSM-5 zeolite with 5 to 95 wt. % silica, clay and/or alumina binder.

These siliceous zeolites can be employed in their acid forms, ion-exchanged or impregnated with one or more suitable metals, such as Ga, Pd, Zn, Ni, Co, Mo, P, and/or other metals of Periodic Groups III to VIII. The zeolite may include other components, generally one or more metals of group IB, IIB, IIIB, VA, VIA or VIIIA of the Periodic Table (IUPAC).

Useful hydrogenation components can include the noble metals of Group VIIIA, such as platinum, but other noble metals, such as palladium, gold, silver, rhenium or rhodium, may also be used. Base metal hydrogenation components may also be used, such as nickel, cobalt, molybdenum, tungsten, copper or zinc.

The catalyst materials may include two or more catalytic components which components may be present in admixture or combined in a unitary multifunctional solid particle.

In addition to the preferred aluminosilicates, the gallosilicate, ferrosilicate and “silicalite” materials may be employed. ZSM-5 zeolites can be useful in the process because of their regenerability, long life and stability under the extreme conditions of operation. Usually the zeolite crystals have a crystal size from about 0.01 to over 2 microns or more, such as 0.02-1 micron.

In various aspects, the catalyst particles can contain about 25 wt. % to about 40 wt. % H-ZSM-5 zeolite, based on total catalyst weight, contained within a silica-alumina matrix. Typical Alpha values for the catalyst can be about 100 or less. Sulfur conversion to hydrogen sulfide can increase as the alpha value increases.

The Alpha Test is described in U.S. Pat. 3,354,078, and in the Journal of Catalysis, Vol. 4, p. 527 (1965); Vol. 6, p. 278 (1966); and Vol. 61, p. 395 (1980), each incorporated herein by reference as to that description.

In various aspects, the hydrocarbon feed may be exposed to the acidic catalyst by using a moving or fluid catalyst bed reactor. In such aspects, the catalyst may be regenerated, such via continuous oxidative regeneration. The extent of coke loading on the catalyst can then be continuously controlled by varying the severity and/or the frequency of regeneration. In a turbulent fluidized catalyst bed, the conversion reactions are conducted in a vertical reactor column by passing hot reactant vapor upwardly through the reaction zone and/or reaction vessel at a velocity greater than dense bed transition velocity and less than transport velocity for the average catalyst particle. A continuous process is operated by withdrawing a portion of coked catalyst from the reaction zone and/or reaction vessel, oxidatively regenerating the withdrawn catalyst and returning regenerated catalyst to the reaction zone at a rate to control catalyst activity and reaction severity to affect feedstock conversion. Preferred fluid bed reactor systems are described in Avidan et al U.S. Pat. No. 4,547,616; Harandi & Owen U.S. Pat. No. 4,751,338; and in Tabak et al U.S. Pat. No. 4,579,999, incorporated herein by reference. In other aspects, other types of reactors can be used, such as fixed bed reactors, riser reactors, fluid bed reactors, and/or moving bed reactors.

In one or more aspects, effective conversion conditions for exposing the hydrocarbon feed to an acidic catalyst can include a temperature of about 300° F. (149° C.) to about 900° F. (482° C.), or about 350° F. (177° C.) to about 850° F. (454° C.), or about 350° F. (177° C.) to about 800° F. (427° C.), or about 350° F. (177° C.) to about 750° F. (399° C.), or about 350° F. (177° C.) to about 700° F. (371° C.), or a temperature of at least about 400° F. (204° C.), or at least about 500° F. (260° C.), or at least about 550° F. (288° C.), or at least about 600° F. (316° C.); a pressure of about 50 psig (0.34 MPag) to about 1100 psig (7.6 MPag), or a pressure of about 100 psig (0.69 MPag) to about 1000 psig (6.9 MPag), or a pressure of about 100 psig (0.69 MPag) to about 200 psig (1.4 MPag), or about 150 psig (1.0 MPag) to about 975 psig (6.7 MPag), or about 200 psig (1.4 MPag) to about 950 psig (6.6 MPag), or about 250 psig (1.7 MPag) to about 900 psig (6.2 MPag), or about 300 psig (4.1 MPag) to about 850 psig (5.9 MPag), or about 300 psig (4.1 MPag) to about 800 psig (5.5 MPag), or a pressure of at least about 50 psig (0.34 MPag), or a pressure of at least about 100 psig (0.69 MPag), or a pressure of at least about 150 psig (1.0 MPag), or a pressure of at least about 200 psig (1.4 MPag), or a pressure of at least about 250 psig (1.7 MPag), or a pressure of at least about 300 psig (4.1 MPag), or a pressure of at least about 350 psig (2.4 MPag); and a total feed WHSV of about 0.05 hr-1 to about 40 hr-1, or about 0.05 to about 30 hr-1, or about 0.1 to about 20 hr-1, or about 0.1 to about 10 hr-1. Optionally, the total feed WHSV can be about 1 hr-1 to about 40 hr-1 to improve C5+ yield.

In addition to a total feed WHSV, a WHSV can also be specified for just the olefin compounds in the feed. In other words, an olefin WHSV represents a space velocity defined by just the weight of olefins in a feed relative to the weight of catalyst. In one or more aspects, the effective conversion conditions can include an olefin WHSV of at least about 0.8 hr-1, or at least about 1.0 hr-1, or at least about 2.0 hr-1, or at least about 3.0 hr-1, or at least about 4.0 hr-1, or at least about 5.0 hr-1, or at least about 8.0 hr-1, or at least about 10 hr-1, or at least about 15 hr-1. In the same or alternative aspects, the effective conversion conditions can include an olefin WHSV of about 40 hr-1 or less, or about 30 hr-1 or less, or about 20 hr-1 or less. In certain aspects, the effective conversion conditions can include an olefin WHSV of about 0.8 hr-1 to about 30 hr-1, or about 0.8 hr-1 to about 20 hr-1, or about 0.8 hr-1 to about 15 hr-1, or about 0.8 hr-1 to about 10 hr-1, or about 0.8 hr-1 to about 7 hr-1, or about 0.8 hr-1 to about 5 hr-1, or about 1.0 hr-1 to about 30 hr-1, or about 1.0 hr-1 to about 20 hr-1, or about 1.0 hr-1 to about 15 hr-1, or about 1.0 hr-1 to about 10 hr-1, or about 1.0 hr-1 to about 7 hr-1, or about 1.0 hr-1 to about 5 hr-1, or about 2.0 hr-1 to about 30 hr-1, or about 2.0 hr-1 to about 20 hr-1, or about 2.0 hr-1 to about 15 hr-1, or about 2.0 hr-1 to about 10 hr-1, or about 2.0 hr-1 to about 7 hr-1, or about 2.0 hr-1 to about 5 hr-1, about 4.0 hr-1 to about 30 hr-1, or about 4.0 hr-1 to about 20 hr-1, or about 4.0 hr-1 to about 15 hr-1, or about 4.0 hr-1 to about 10 hr-1, or about 4.0 hr-1 to about 7 hr-1. An olefin WHSV of about 1 hr-1 to about 40 hr-1 can be beneficial for increasing the C5+ yield.

In various aspects, decreasing the temperature when the olefin WHSV is increased, e.g., when the olefin WHSV is increased above 1 hr-1, may improve product yield. For example, in such aspects, temperatures of about 600° F. (316° C.) to about 800° F. (427° C.), or about 650° F. (343° C.) to about 750° F. (399° C.) may aid in increasing product yield, such as the yield of C5+ compounds, when the olefin WHSV is increased above 1 hr-1.

### Regeneration

The catalyst is regenerated to burn coke that is formed and deposited on the catalyst during oligomerization reactions. In embodiments, air may be supplied to the regenerator in about stoichiometric or less than stoichiometric amounts to produce carbon dioxide and carbon monoixide. For example, the regeneration may be conducted with less than 0.05% stoichiometric excess oxygen, inclusive of a stoichiometric oxygen defecit. In such an embodiment, the regenerator may be reduced to a pipe that withdraws catalyst from the reactor and the regeneration zone is maintained within the pipe to burn the coke. The catalyst may eventually be lifted to the reactor using the feed fuel gas.

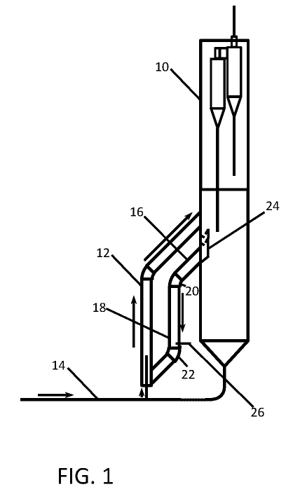

As illustrated in FIG. 1, the reactor 10 includes a lift leg 12 for receiving a portion of the feed 14 (e.g., about 5-10 wt % of the total hydrocarbon feed to the process) and regenerated catalyst particles. The catalyst particles are regenerated in regeneration leg 16, which includes a regeneration zone 18 which may be in the form of a vertical pipe (e.g., a reactor stand-pipe) defined on one or both ends by a bend 20, 22. On the other side of bend 20 is an inclined section of pipe connecting the regeneration zone 18 with an optional stripper 24 within reactor 10. In such a case, the regeneration gas (or combustion by-products thereof) may also function to some extent as a stripping gas. Steam or other stripping gas my also be employed in the stripper 24. On the other side of bend 22, is an inclined section of pipe that is fluidly connected to the lift leg 12 allowing regenerated particles to combine with a portion of feed 14 and be returned to the reactor 10. At or near the end of the regenerator zone 18 proximal bend 22, combustion air is fed to the regeneration zone 18 by air sparger 26.

The regeneration zone 18 may be much smaller than typical regenerator vessels. For example, the diameter of the pipe in the regeneration zone may be less than 5 feet in diameter, such as less than 4 feet in diameter, or less than 3 feet in diameter, or less than or equal to 2 feet in diameter, such as about 1 to about 2 feet in diameter. In addition, the regeneration zone may be less than 50 feet in length, such as less than 40 feet in length, such as less than 30 feet in length, such as from about 5 feet to about 30 feet in length, such as about 5 feet to about 25 feet in length. The regneration vessel itself can comprise refractory liner (e.g., a “cold-wall” material).

In such embodiments, the reduced regeneration air requirements allow for reduced air compression and air heating, allowing for the physical size, cost and utility demands on such equipment to be reduced. Furthermore, the compressor may be eliminated if a suitable source of air is available.

Although not shown in the current simplified schematic, nitrogen can be used during startup and shutdown to provide the lift for the catalyst particles in the lift leg 12. During regular operation, a portion of the feed to the reactor 10, such as 1 to 15 volume %, is routed to the lift leg 12 to carry the regenerated catalyst to the reactor 10.

Optionally, an interlock system may be employed and activated to cut off air to the regeneration leg 16 when temperatures in the regeneration zone 18 exceed a designated value, such as 1200° F. or 1400° F. or when temperatures in the regeneration zone 18 falls below a certain value, such as 750° F. or 700° F.

### Embodiments

In addition to the foregoing, the following embodiments are also considered:

### Embodiment 1

A method of upgrading a hydrocarbon feed comprising: contacting a hydrocarbon feed with a catalyst in a fluidized bed reactor; directing a portion of the catalyst from the fluidized bed reactor to a regeneration zone, such that the portion of the catalyst flows in a first direction through the regeneration zone; directing combustion air into the regeneration zone in a counter-flow direction to the first direction, wherein the combustion air is provided at a rate of about 100.05% or less of the stoichiometric air requirement for combusting coke present on the portion of catalyst; regenerating the portion of the catalyst in the regeneration zone to produce regenerated catalyst; and directing a portion of the hydrocarbon feed to combine with the regenerated catalyst downstream of the regeneration zone and lift the regenerated catalyst back to the fluidized bed reactor.

### Embodiment 2

The method of any other enumerated Embodiment, wherein the rate of combustion air is less than 100% of the stoichiometric air requirement.

### Embodiment 3

The method of any other enumerated Embodiment, wherein the regenerated catalyst is fed to a lift leg where it combines with the portion of the fuel gas.

### Embodiment 4

The method of any other enumerated Embodiment, wherein the portion of the catalyst is gravity fed through regeneration zone.

### Embodiment 5

The method of any other enumerated Embodiment, wherein the regeneration zone is a vertically-oriented conduit and the first direction is a vertical direction.

### Embodiment 6

The method of any other enumerated Embodiment, wherein the portion of the hydrocarbon feed is less than about 20 wt % of the hydrocarbon feed fed to the fluidized bed reactor.

### Embodiment 7

The method of any other enumerated Embodiment, wherein the portion of the hydrocarbon feed is between about 5 wt % and about 10 wt % of the hydrocarbon feed fed to the fluidized bed reactor.

### Embodiment 8

The method of any other enumerated Embodiment, further comprising directing byproducts of the combustion of coke into the fluidized bed reactor.

### Embodiment 9

The method of any other enumerated Embodiment, further comprising directing byproducts of the combustion of coke into a stripping section within the fluidized bed reactor.

### Embodiment 10

The method of any other enumerated Embodiment, wherein the step of contacting a hydrocarbon feed with a catalyst comprises converting a fuel gas to gasoline boiling range hydrocarbons.

### Embodiment 11

The method of any other enumerated Embodiment, wherein the step of contacting a hydrocarbon feed with a catalyst comprises reacting sulfur compounds in a fluid catalytic cracking naphtha feed.

### Embodiment 12

The method of any other enumerated Embodiment, wherein the catalyst is ZSM-5.

### Embodiment 13

A system for upgrading a hydrocarbon feed, such as a fuel gas comprising: a hydrocarbon feed, such as a fuel gas feed; a fluidized bed reactor containing a catalyst for upgrading the hydrocarbon feed (e.g., converting the fuel gas to gasoline boiling range hydrocarbons) ; a regeneration leg fluidly connected with the fluidized bed reactor for receiving a portion of catalyst to be regenerated and permit the portion of catalyst to flow in a first direction through a regeneration zone; and a combustion air feed fluidly connected with the regeneration leg and adapted to inject combustion air into the regeneration leg so that the combustion air flows through the regeneration zone in a counter-flow direction to the first direction to produce a regenerated catalyst; a lift leg fluidly connected to the regeneration leg to receive the regenerated catalyst, the lift leg further fluidly connected to the fuel gas feed to receive a portion of the fuel gas feed to lift the regeneration catalyst away from the regeneration leg and return the regenerated catalyst to the fluidized bed reactor.

### Embodiment 14

The system of any enumerated Embodiment, wherein the regeneration zone is contained within a vertically-oriented stand-pipe.

### Embodiment 15

The system of any enumerated Embodiment, wherein the stand-pipe has an inner diameter of less than 4 feet.

### Embodiment 16

The system of any enumerated Embodiment, wherein the inner diameter is less than or equal to 2 feet.

### Embodiment 17

The system of any enumerated Embodiment, wherein the regeneration zone is about 5 feet to about 30 feet in height.

1. A method of upgrading a hydrocarbon feed comprising:
contacting a hydrocarbon feed with a catalyst in a fluidized bed reactor;
directing a portion of the catalyst from the fluidized bed reactor to a regeneration zone, such that the portion of the catalyst flows in a first direction through the regeneration zone;
directing combustion air into the regeneration zone in a counter-flow direction to the first direction, wherein the combustion air is provided at a rate of about 100.05% or less of the stoichiometric air requirement for combusting coke present on the portion of catalyst;
regenerating the portion of the catalyst in the regeneration zone to produce regenerated catalyst; and
directing a portion of the hydrocarbon feed to combine with the regenerated catalyst downstream of the regeneration zone and lift the regenerated catalyst back to the fluidized bed reactor.

2. The method of claim 1, wherein the rate of combustion air is less than 100% of the stoichiometric air requirement.

3. The method of claim 1, wherein the regenerated catalyst is fed to a lift leg where it combines with the portion of the fuel gas.

4. The method of claim 1, wherein the portion of the catalyst is gravity fed through regeneration zone.

5. The method of claim 1, wherein the regeneration zone is a vertically-oriented conduit and the first direction is a vertical direction.

6. The method of claim 1, wherein the portion of the hydrocarbon feed is less than about 20 wt % of the hydrocarbon feed fed to the fluidized bed reactor.

7. The method of claim 6, wherein the portion of the hydrocarbon feed is between about 5 wt % and about 10 wt % of the hydrocarbon feed fed to the fluidized bed reactor.

8. The method of claim 1, further comprising directing byproducts of the combustion of coke into the fluidized bed reactor.

9. The method of claim 1, further comprising directing byproducts of the combustion of coke into a stripping section within the fluidized bed reactor.

10. The method of claim 1, wherein the step of contacting a hydrocarbon feed with a catalyst comprises converting a fuel gas to gasoline boiling range hydrocarbons.

11. The method of claim 1, wherein the step of contacting a hydrocarbon feed with a catalyst comprises reacting sulfur compounds in a fluid catalytic cracking naphtha feed.

12. The method of claim 1, wherein the catalyst is ZSM-5.

13. A system for upgrading a fuel gas comprising:
a fuel gas feed;
a fluidized bed reactor containing a catalyst for converting the fuel gas to gasoline boiling range hydrocarbons;
a regeneration leg fluidly connected with the fluidized bed reactor for receiving a portion of catalyst to be regenerated and permit the portion of catalyst to flow in a first direction through a regeneration zone; and
a combustion air feed fluidly connected with the regeneration leg and adapted to inject combustion air into the regeneration leg so that the combustion air flows through the regeneration zone in a counter-flow direction to the first direction to produce a regenerated catalyst;
a lift leg fluidly connected to the regeneration leg to receive the regenerated catalyst, the lift leg further fluidly connected to the fuel gas feed to receive a portion of the fuel gas feed to lift the regeneration catalyst away from the regeneration leg and return the regenerated catalyst to the fluidized bed reactor.

14. The system of claim 13, wherein the regeneration zone is contained within a vertically-oriented stand-pipe.

15. The system of claim 14, wherein the stand-pipe has an inner diameter of less than 4 feet.

16. The system of claim 15, wherein the inner diameter is less than or equal to 2 feet.

17. The system of claim 13, wherein the regeneration zone is about 5 feet to about 30 feet in height.
